(19) United States
(12) Patent Application Publication
(10) Pub. No.: US 2011/0130285 A1
(43) Pub. Date: Jun. 2, 2011

(54) COMPOSITION FOR PROMOTING ROOT NODULE FORMATION OF PLANTS AND A METHOD FOR PROMOTING ROOT NODULE FORMATION OF PLANTS

(76) Inventors: Kazuhiko Watanabe, Takasago-shi (JP); Daisuke Yokoi, Kawasaki-shi (JP)

(21) Appl. No.: 12/956,263

(22) Filed: Nov. 30, 2010

(30) Foreign Application Priority Data
Nov. 30, 2009 (JP) 2009-272363
Jun. 21, 2010 (JP) 2010-140639

(51) Int. Cl. A01N 43/90; C07H 19/167; C07H 19/067; C07H 19/20; C07H 19/10; C07D 473/30; C07D 473/18; C07D 239/54; A01N 43/54; A01N 57/16; A01P 21/00

(52) U.S. Cl. 504/196; 536/27.8; 536/27.81; 536/28.53; 536/26.74; 536/26.7; 536/26.8; 544/265; 544/276; 544/309; 504/241; 504/243

(57) ABSTRACT

A composition for promoting root nodule formation, which can be easily applied to soil or plants, and enables efficient formation of root nodules, and a method for promoting root nodule formation using the composition are provided. A composition for promoting root nodule formation, which comprises a compound selected from the group consisting of a nucleoside, a nucleotide, and a nucleobase such as inosine, as an active ingredient, is applied to a leguminous plant to promote root nodule formation of the plant.

This application claims priority under 35 U.S.C. §119 to Japanese Patent Application Nos. 2009-272363, filed on Nov. 30, 2009, and 2010-140639, filed on Jun. 21, 2010, which are both incorporated in their entireties by reference.

### BACKGROUND OF THE INVENTION

1. Field of the Invention

The present invention relates to a composition for promoting formation of root nodules in plant roots, and a method for promoting formation of root nodules.

2. Brief Description of the Related Art

Many nutrients required for growth of plants are applied as fertilizers. Although nitrogenous fertilizers constitute one class of typical fertilizers, their production requires a lot of energy, and there is concern about their effect on the environment. For example, use of large amounts of nitrogenous fertilizers may pollute groundwater, and the fertilizers may vaporize as N₂O gas, which is a cause of global warming.

Moreover, leguminous plants can grow in the absence of nitrogenous fertilizer. Root nodule bacteria, a kind of soil bacteria, enters into the roots of leguminous plants and forms root nodules to obtain carbohydrates from the plants and fix nitrogen in the air to give nitrogenous compounds to the plants. Thus, leguminous plants and root nodule bacteria are in a symbiotic relationship. Growth of plants is greatly influenced by the degree of formation of root nodules of the plants. With sufficient formation of root nodules, leaves and stems extend well, and flower buds are well formed to induce fertilization, fructification and fating of fruits.

Methods of artificially inoculating root nodule bacteria into leguminous plants have been attempted as methods for promoting formation of root nodules, (Japanese Patent Laid-open (Kokai) Nos. 3-266915, 6-62667, 6-141848, 8-109109, and 8-109110). However, all of these methods require a carrier for the root nodule bacteria, and these may not be the most satisfactory or economic methods and may be labor intensive. Furthermore, although a method of coating plant seeds with powdery root nodule bacteria and seeding them has also been proposed (Japanese Patent Laid-open No. 10-210807), this may not be the simplest method available. Moreover, under these methods the inoculated root nodule bacteria may not preferentially adhere to plants in a viable state and the distribution of the inoculated root nodule bacteria is limited.

Additionally, a technique for improving viable adhesion of root nodule bacteria has been described, including a method of applying an inoculation material obtained by adding a betaine compound to a root nodule bacteria suspension to seeds of leguminous plants (Japanese Patent Laid-open No. 2003-40720). However, this may not be the simplest method either.

Inosine is known to promote the growth of plant roots when it is applied to soil or hydroponic water (Japanese Patent No. 2927269). However, so far this application has been limited to leaf vegetables, fruit vegetables, root vegetables, flowers and fruit trees, and therefore, the influence of inosine on root nodule formation of leguminous plants is still uncertain.

What's more, a nucleic acid material from a decomposing yeast cell extract, that is collected by methods of alkaline degradation and enzymolysis has been described, and concentrating the resultant product increases the activity of nitrogen-fixing enzymes of leguminous plants and bean number of soybean plants (Min, S. et al., “Effects on leguminaceous crop nodule bacteria nitrogenase activity with nucleotide”, Weishengwuxue Zazhi, vol. 10, pp. 58-60, 1990, 4th term). However, this described nucleic acid material is in the form of a paste, and therefore, it is not considered to consist of low molecule nucleic acids.

### SUMMARY OF THE INVENTION

An aspect of the present invention is to provide a composition for promoting root nodule formation, which can be applied easily to soil or plants, and enables efficient formation of root nodules. A further aspect of the present invention is to provide a method for promoting root nodule formation using such a composition.

These aspects were based on the findings that nucleosides, nucleotides and nucleobases such as inosine, had an effect of promoting formation of root nodules of plants.

Accordingly, it is an aspect of the present invention to provide a composition for promoting root nodule formation of a leguminous plant, which comprises a compound selected from the group consisting of a nucleoside, a nucleotide, and a nucleobase as an active ingredient.

It is a further aspect of the present invention to provide the composition for promoting root nodule formation as described above, which contains a fermentation by-product comprising the compound.

It is a further aspect of the present invention to provide the composition for promoting root nodule formation as described above, wherein the compound is selected from the group consisting of inosine, guanosine, uridine, inosinic acid, guanylic acid, uridylic acid, hypoxanthine, guanine, and uracil.

It is a further aspect of the present invention to provide the composition for promoting root nodule formation as described above, wherein the compound is selected from the group consisting of inosine, guanosine, hypoxanthine, and uracil.

It is a further aspect of the present invention to provide the composition for promoting root nodule formation as described above, which is applied to soil or a plant in an amount of 0.1 g to 20 kg/ha in terms of nucleoside.

It is a further aspect of the present invention to provide the composition for promoting root nodule formation as described above, which is in the form of a liquid containing 0.01 to 100 ppm of the compound in terms of nucleoside at the time of use.

It is a further aspect of the present invention to provide a method for promoting root nodule formation of a leguminous plant, which comprises applying the composition for promoting root nodule formation as described above, to soil or the plant.

It is a further aspect of the present invention to provide the method as described above, wherein the plant is a soybean plant.

It is a further aspect of the present invention to provide the method as described above, wherein the composition for promoting root nodule formation as described above, is applied to soil by spraying on a soil surface or by irrigation into soil, or to the plant by foliar spray.

It is a further aspect of the present invention to provide the method as described above, wherein the composition for promoting root nodule formation is applied in an amount of 0.1 g to 20 kg/ha in terms of nucleoside.

It is a further aspect of the present invention to provide the method as described above, wherein the composition for promoting root nodule formation is applied as an aqueous solution at a concentration of 0.01 to 100 ppm in terms of nucleoside.

It is a further aspect of the present invention to provide the method as described above, wherein the compound is selected from the group consisting of inosine, guanosine, uridine, inosinic acid, guanylic acid, uridylic acid, hypoxanthine, guanine, and uracil.

It is a further aspect of the present invention to provide the method as described above, wherein the compound is selected from the group consisting of inosine, guanosine, hypoxanthine, and uracil.

According to the present invention, formation of root nodules of leguminous plants can be promoted. As a result, growth of the leguminous plants can be improved, and the consumption of nitrogenous fertilizer can be reduced. The composition for promoting root nodule formation of the present invention does not need any specific carrier other than the nucleoside, nucleotide or nucleobase. Moreover, the method for promoting root nodule formation of the present invention is convenient since it does not require a complicated operation at the time of application.

### BRIEF DESCRIPTION OF THE DRAWINGS

FIG. 1 is a graph showing the number of root nodules of soybeans grown for 20 days after seeding with an application of inosine by spraying on the soil.

FIG. 2 is a graph showing the dry weight of aboveground parts of soybeans grown for 20 days after seeding with an application of inosine by spraying on the soil.

FIG. 3 is a graph showing the number of root nodules of soybeans grown for 34 days after seeding with an application of inosine by irrigation into the soil.

FIG. 4 is a graph showing the dry weight of root nodules of soybeans grown for 34 days after seeding with an application of inosine by irrigation into the soil (relative values based on the value of the distilled water-applied group, which is taken as 100).

FIG. 5 is a graph showing the number of root nodules of soybeans grown for 32 days after seeding with an application of inosine by foliar spray.

FIG. 6 is a graph showing the dry weight of root nodules of soybeans grown for 32 days after seeding with an application of inosine by foliar spray (relative values based on the value of the distilled water-applied group, which is taken as 100).

FIG. 7 is a graph showing the number of root nodules of soybeans grown for 33 days after seeding with an application of inosine by irrigation into the soil.

FIG. 8 is a graph showing the dry weight of root nodules of soybeans grown for 33 days after seeding with an application of inosine by irrigation into the soil.

FIG. 9 is a graph showing the dry weight of aboveground parts of soybeans grown for 33 days after seeding with an application of inosine by irrigation into the soil.

FIG. 10 is a graph showing the dry weight of underground parts of soybeans grown for 33 days after seeding with an application of inosine by irrigation into the soil.

FIG. 11 is a graph showing the number of root nodules of soybeans grown for 40 days after seeding with an application of various compounds or fermentation by-product solutions by irrigation into the soil.

FIG. 12 is a graph showing the dry weight of root nodules of soybeans grown for 40 days after seeding with an application of various compounds or fermentation by-product solutions by irrigation into the soil.

FIG. 13 is a graph showing the dry weight of aboveground parts of soybeans grown for 33 days after seeding with an application of various compounds or fermentation by-product solutions by irrigation into the soil.

FIG. 14 is a graph showing the dry weight of underground parts of soybeans grown for 33 days after seeding with an application of various compounds or fermentation by-product solutions by irrigation into the soil.

### DETAILED DESCRIPTION OF EXEMPLARY EMBODIMENTS

Hereafter, the present invention will be explained in detail.

A composition for promoting root nodule formation in accordance with the presently disclosed subject matter includes a compound such as a nucleoside, a nucleotide and a nucleobase as an active ingredient.

The nucleobase may be a purine base or a pyrimidine base. The nucleoside may be a purine nucleoside or a pyrimidine nucleoside, and the nucleotide may be a purine nucleotide or a pyrimidine nucleotide. The saccharide constituting the nucleoside or nucleotide may be ribose or deoxyribose.

Examples of the nucleobase include, but are not limited to, adenine, guanine, thymine, cytosine, uracil, xanthine, and hypoxanthine. Specifically, one example of the nucleobase may be a purine base, or hypoxanthine, guanine, and uracil in another example.

Examples of the nucleoside include, but are not limited to, adenosine, guanosine, thymidine, cytidine, uridine, xanthosine, inosine, and 2′-deoxy compounds thereof. Specifically, the nucleoside may be, for example, inosine, guanosine, and uracil.

Examples of the nucleotide include, but are not limited to, adenylic acid (adenosine 5′-phosphate), guanylic acid (guanosine 5′-phosphate), thymidylic acid (thymidine 5′-phosphate), uridylic acid (uridine 5′-phosphate), xanthylic acid (xanthosine 5′-phosphate), inosinic acid (inosine 5′-phosphate), and 2′-deoxy compounds thereof. Specifically, the nucleotide may be, for example, inosinic acid and guanylic acid, or uridylic acid in another example.

The nucleoside, nucleotide and nucleobase may exist in free forms or salts, such as sodium salts and potassium salts.

The composition for promoting root nodule formation may contain one kind of compound such as a nucleoside, a nucleotide and a nucleobase, or two or more kinds of such compounds.

The compound may be a purified product, or the compound may be in the form of a composition containing the above-described compound, so long as the formation of root nodules and the growth of plants to which the composition is applied are not adversely affected. Examples of such a composition include, but are not limited to, fermentation liquor, concentrate thereof, dried product of concentrate, roughly purified product, fermentation by-product, or fractionation product thereof.

Nucleosides, nucleotides and nucleobases can be produced, for example, by the methods described in Japanese Patent Laid-open Nos. 11-346778, 2004-242610, and 2007-105055.

The fermentation by-product may be a fermentation solution obtained by removing a major product which is a target substance of the fermentation from the fermentation medium (also referred to as “fermentation by-product solution”), a concentrate or dried product thereof, or fractionation products thereof, so long as, when the fermentation by-product solution is applied to a leguminous plant, it can promote formation of root nodules. An acid or the like may be added to the fermentation by-product solution in order to collect an objective product from a medium, or the fermentation by-product solution may be heated for bacteria elimination or the like.

Examples of the fermentation by-product solution containing the compound include, but are not limited to, a fermentation by-product solution obtained by culturing a microorganism which produces the compound in a medium containing any of a variety of carbohydrate materials such as molasses, tapioca and corn as a sugar source, and any of a variety of ammonia nitrogen materials such as ammonia and ammonium sulfate, separating and removing nucleic acids from the resulting fermentation solution. Examples of the microorganism and the medium include, but are not limited to, those described on Japanese Patent Laid-open Nos. 11-346778, 2004-242610, 2007-105055. Specific examples of the fermentation by-product solution include, but are not limited to, a mother solution obtained by adjusting the pH of a fermentation solution containing the compound, to the isoelectric point of the compound with a mineral acid such as sulfuric acid or hydrochloric acid, and/or cooling such a fermentation solution to precipitate the compound, and separating the solid from the fermentation solution, and concentrate thereof. The obtained fermentation by-product solution typically contains, in addition to the compound, an abundance of volatile basic nitrogens, nucleic acid organic nitrogens, amino acid organic nitrogens, and nitrogen derived from fermented microbial cells. Moreover, the fermentation by-product solution contains an abundance of minerals including sulfur and chlorine derived from mineral acid used at the time of adjusting the pH of a nucleic acid fermentation solution. Additionally, the fermentation by-product solution contains trace components including, but not limited to, minerals other than those described above, vitamins, saccharides, organic acids, and fermented microbial cells.

The method for applying the composition for promoting root nodule formation is not particularly limited, so long as the composition is brought into contact with, or delivered to, plant bodies or soil around roots of plants, through means including but not limited to, for example, spraying on the soil surface, irrigation into soil, plowing-in the soil, foliar spray on plants, applying as a mixture with fertilizer, or as an addition to hydroponic solution.

The form of the composition for promoting root nodule formation is not particularly limited, and it can be applied in the forms of conventional materials for agriculture and horticulture. For example, the composition can be applied in a common form such as a solid, powder or liquid, according to the types of the application methods described above.

The composition for promoting root nodule formation may contain additional components other than the compound. For example, these components include, but are not limited to, solvents, carriers, pH modifiers for promoting dissolution of the compound, spreading agent for enhancing spread of the composition on plant bodies or soil, fertilizers for enhancing fertilizing effect, agrochemical components, binders, and fillers.

Examples of the solvent include water and so forth. Examples of the carrier include, but are not limited to, diatomaceous earth, vermiculite, perlite, peat moss, activated carbon, and humus. However, the composition for promoting root nodule formation does not necessarily require a specific carrier.

Upon use, the composition for promoting root nodule formation in the solid form may be dissolved or dispersed in a solvent such as water. The composition may also be used with the addition of a spreading composition comprising a surfactant and so forth, or as a mixture with agricultural chemicals.

The composition for promoting root nodule formation may further contain nucleic acids other than nucleosides and nucleotides, such as, for example, oligonucleotides and polynucleotides, so long as the above-described compound is contained in an effective amount. The composition may also contain conventionally known components having a root nodule formation promotion action. The composition may additionally contain other microorganisms such as Azospirillum bacteria. The composition may further contain root nodule bacteria.

The content of the nucleoside, nucleotide or nucleobase in the composition for promoting root nodule formation is not particularly limited, and can be apportioned so that it is suitable for the application amount described below. For example, in the case of an aqueous solution, the total content of the nucleoside, nucleotide and nucleobase can be 0.01 to 100 ppm, 0.5 to 50 ppm, or 2 to 20 ppm.

By applying the above-described composition for promoting root nodule formation to soil or plants, the formation of root nodules of plants can be promoted. The promotion of the formation of root nodules includes increasing the number of root nodules, increasing the weight of each root nodule, and a combination thereof. As a result of the promotion of the formation of root nodules, growth is promoted of the aboveground parts and/or the underground parts of plants, and an increase in weight, especially an increase in the yield of beans occurs.

The soil is not particularly limited, and the composition for promoting root nodule formation exhibits a marked root nodule formation promoting effect in both low nutrient soil and high nutrient soil. However, the composition exhibits an especially marked root nodule formation promoting effect in low nutrient soil.

The plant is not particularly limited, so long as it is a plant that can form root nodules. For example, leguminous plants are generally known to form root nodules. Examples of leguminous plants include, but are not limited to, soybean, adzuki bean (Phaseolus angularis), broad bean (Vicia faba), garden pea (Pisum sativum), peanut, cowpea (Vigna sinensis), lupin bean, clover, and alfalfa. The variety of soybean plants is not particularly limited, so long as root nodules are formed, and the compound promotes root nodule formation. Examples of soybean plants include, but are not limited to, Tanba-kuro, Chusei-hikari-kuro, and Sachiyutaka. The plant may undergo genetic recombination such as genetic recombination for impartation of herbicide resistance.

The root nodule bacterium is not particularly limited, so long as it is a bacterium that can symbiotically grow with a plant to form root nodules, and that formation of root nodules is promoted by the above-described composition for promoting root nodule formation. Examples of the root nodule bacterium include, but are not limited to, bacteria belonging to the genus Rhizobium, Bradyrhizobium, Sinorhizobium, and Mesorhizobium. Specific examples include, but are not limited to, Rhizobium leguminosarum, Rhizobium tropici, Shinorhizobium meliloti, Sinorhizobium fredii, Bradyrhizobium japonicum, Bradyrhizobium elkani, Mesorhizobium loti, and Mesorhizobium huakuii.

Although the application amount of the composition for promoting root nodule formation may vary depending on the application method, application stage, type of plant, cultivation density, growth stage, and so forth, for one season it can be 0.1 g/ha to 20 kg/ha or 2 g/ha to 2 kg/ha, in terms of nucleoside. The “amount in terms of nucleoside” means the amount of corresponding nucleoside in the case of a nucleotide or nucleobase, or the amount of inosine in the case of hypoxanthine and inosinic acid, for example.

The composition for promoting root nodule formation may be applied at one time in an amount in the above-described range, or applied two or more times in divided portions of such an amount of the composition.

Although the frequency and quantity of the applications may vary depending on the type of the plant, etc., the composition is usually applied once or two to four times, during 3 to 60 days, or 5 to 30 days, after seeding, in the case of soybean, for example.

In addition to the application of the composition for promoting root nodule formation, root nodule bacteria may be inoculated into the soil or the seeds, or mineral materials such as molybdenum, cobalt and iron may be used together.

### EXAMPLES

Hereinafter, the present invention will be explained more specifically with reference to the following non-limiting examples.

### Example 1

### Effect of Inosine on Root Nodule Formation of Soybean

Various varieties of soybeans (black soybeans: Tanba-kuro and Chusei-hikari-kuro, white soybean: Sachiyutaka) were each seeded in 0.6 L of soil (marketed vermiculite, fertilizer was not added) contained in each of No. 4 pots (diameter: 12 cm). For each variety, 7 individuals showing good growth before development of primary leaf were selected per pot. Seven individuals were used for each of the following treatment groups.

Treatment Groups

1. Distilled water group

2. 2 ppm Inosine solution group

3. 20 ppm Inosine solution group

4. 100 ppm Inosine solution group

Each solution in a volume of 250 ml was splayed on the soil surface in the pot, 7, 10 and 15 days after the seeding. The inosine concentrations in the soil given as a one-time application were 0.83 ppm for the 2 ppm solution group, 8.3 ppm for the 20 ppm solution group, and 41.7 ppm for the 100 ppm solution group, which were equivalent to 442 g/ha, 4,425 g/ha, and 22.1 kg/ha, respectively. The inosine solutions were prepared by diluting a 2% inosine solution (Ikuou, Shoko Agri Co., Ltd., containing 0.52% KOH as pH modifier in order to promote dissolution of inosine) with distilled water.

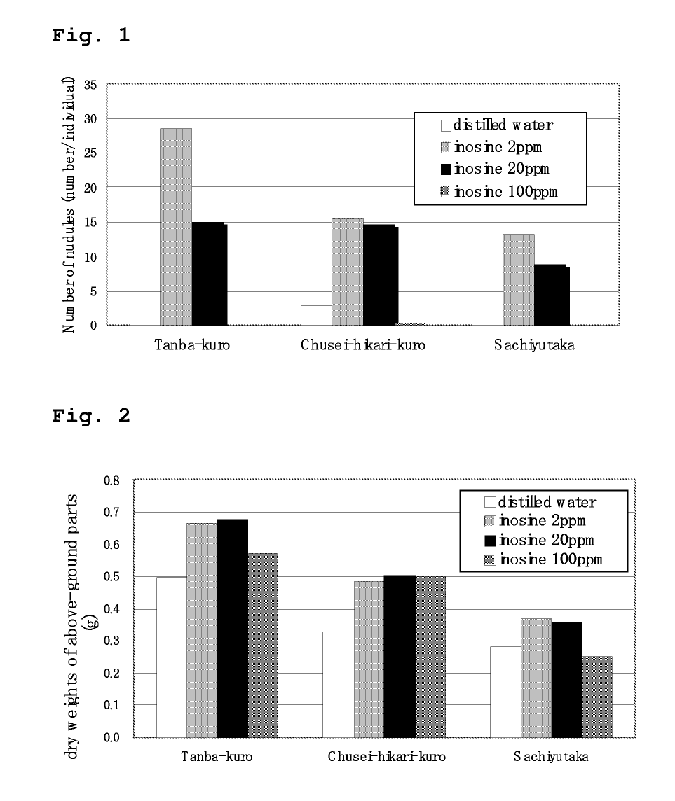

Twenty days after the seeding, the number of root nodules (FIG. 1) and the dry weight of aboveground parts (FIG. 2) were measured. For all the varieties, Tanba-kuro, Chusei-hikari-kuro, and Sachiyutaka, the numbers of root nodules and the dry weights of the aboveground parts clearly increased in the 2 ppm and 20 ppm inosine solution treatment groups.

### Example 2

### Application of Inosine by Irrigation into Soybean-Growing Soil

Soybean (Sachiyutaka) was seeded in 1 kg of soil (marketed black soil, moisture content: 35%) contained in each of the pots. Calcium superphosphate and potassium sulfate were added as fertilizers in the amounts of 40 mg/pot, in terms of P₂O₅ and K₂O, respectively.

The treatment groups included the distilled water group and the 2 ppm inosine solution group. One individual showing good growth before development of a primary leaf was selected from each pot, and five individuals were used for each treatment group. Either the distilled water or the inosine solution in a volume of 250 ml was applied to the soil in the pot by irrigation, 10, 14 and 19 days after the seeding. The inosine concentration in the soil given as a one-time application was 0.5 ppm, which was equivalent to 325 g/ha, in the 2 ppm solution group.

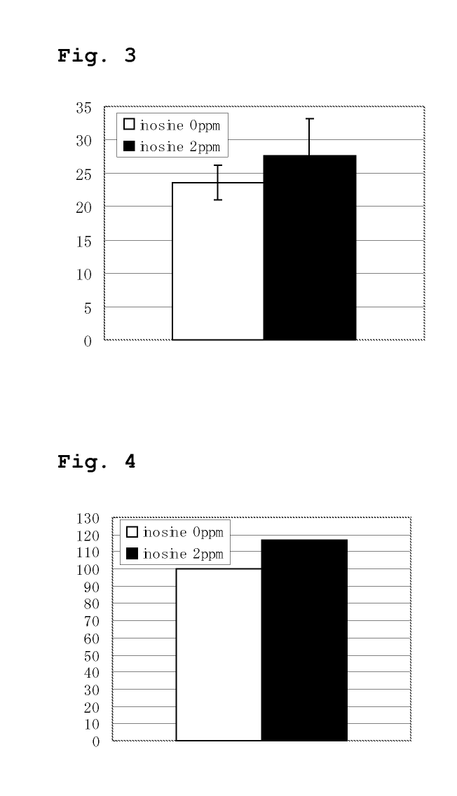

Thirty-four days after the seeding, the number of root nodules (FIG. 3), the dry weight of root nodules (FIG. 4), the dry weight of aboveground parts, and the dry weight of underground parts (Table 1) were measured. The dry weight of root nodules, the dry weight of aboveground parts, and the dry weight of underground parts were indicated with relative values based on the values of the distilled water group, which were taken as 100. The application of the 2 ppm inosine solution increased all of the number of root nodules, the dry weight of root nodules, the dry weight of aboveground parts and the dry weight of underground parts.

TABLE 1

| | Dry weight of | Dry weight of |
|---|---|---|
| Inosine (ppm) | aboveground part | underground part |
| 0 | 100 | 100 |
| 2 | 105.1 | 107.2 |

### Example 3

### Application of Inosine to Soybean by Foliar Splay

Soybean (Sachiyutaka) was seeded in 0.7 kg of soil (marketed black soil, moisture content: 35%) contained in each of pots. Calcium superphosphate and potassium sulfate were added as fertilizers in the amounts of 28 mg/pot, in terms of P₂O₅ and K₂O, respectively. As root nodule bacterium, “Mame-Zo” (containing a soybean root nodule bacterium, Bradyrhizobium japonicum, Tokachi Federation of Agricultural Cooperative Associations, http://www.nokyoren.or.jp/material.html) was added to the soil at a root nodule bacterium concentration of 10⁴ cfu/cm³ of soil.

The treatment groups included the distilled water group and the 10 ppm inosine solution group. One individual showing good growth before development of a primary leaf was selected from each pot, and five individuals were used for each treatment group. Either distilled water or the inosine solution in a volume of 5 ml was applied by foliar spray, 10, 14 and 19 days after the seeding. To the distilled water and the inosine solution, a spreading composition (Approach BI, Kao Corp., “Approach” is a registered trade name of Kao Corp.) was added at a concentration of 0.1 v/v %. The spray amount of inosine given as a one-time application was 10 g/ha in the 10 ppm inosine solution group.

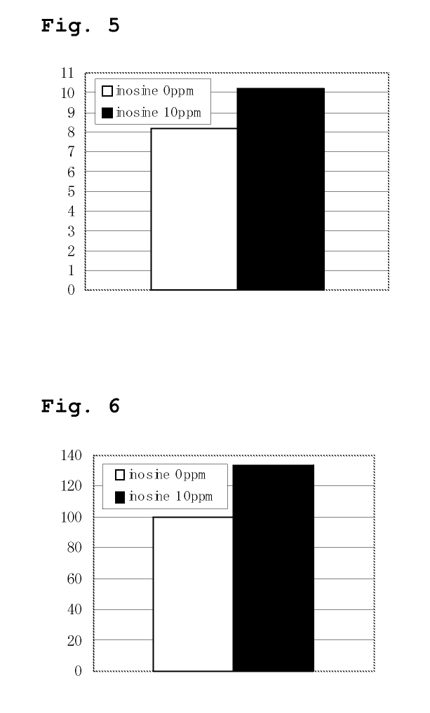

Thirty-two days after the seeding, the number of root nodules (FIG. 5), the dry weight of root nodules (FIG. 6), the dry weight of aboveground parts, and the dry weight of underground parts (Table 2) were measured. The dry weight of root nodules, the dry weight of aboveground parts, and the dry weight of underground parts were indicated with relative values based on the values of the distilled water group, which were taken as 100. The application of the 10 ppm inosine solution increased all of the number of root nodules, the dry weight of root nodules, the dry weight of aboveground parts and the dry weight of underground parts.

TABLE 2

| | Dry weight of | Dry weight of |
|---|---|---|
| Inosine (ppm) | aboveground part | underground part |
| 0 | 100 | 100 |
| 2 | 122.0 | 111.9 |

### Example 4

### Application of Inosine to Soybean-Growing Soil by Irrigation

Soybean (Sachiyutaka) was transplanted to 1.4 L of soil (vermiculite, moisture content: 50%) contained in each of pots (diameter: 15 cm).

The treatment groups included the distilled water groups (Groups 1 and 4) and the inosine application groups (Groups 2, 3, 5 and 6), in which 2 ppm and 20 ppm inosine solutions were applied (Ikuou [Shoko Agri Co., Ltd.] was diluted with distilled water and applied), as shown in Table 3. The distilled water groups and the inosine application groups both include a group in which calcium superphosphate and potassium sulfate were added as fertilizers in amounts of 40 mg/pot in terms of P₂O₅ and K₂O, respectively, and a group in which an organic fertilizer (“Hanasaku Hiryo (1.5-9-4.5)”, Tosho Co., Ltd.) was added in an amount of 2 g/pot (N:P₂O₅:K₂O=30:180:90 mg/pot). The root nodule bacterium (“Mame-Zo”, Tokachi Federation of Agricultural Cooperative Associations) was added at a concentration of 4×10⁴ cfu/cm³ of soil. One individual showing good growth before development of a primary leaf was selected from each pot, and five individuals were used for measurement of each treatment group.

Either distilled water or each of the inosine solutions in a volume of 250 ml was applied by irrigation, 10, 17 and 25 days after the seeding. The inosine concentrations in the soil given as a one-time application were 0.36 ppm for the 2 ppm solution groups, and 3.6 ppm for the 20 ppm solution groups, which were equivalent to 282 g/ha and 2,825 g/ha, respectively. Under the low nutrient soil condition (Groups 1 to 3), etiolation of the leaves was observed and considered to be due to deficiency of trace components. Accordingly, a solution of the composition shown in Table 4 was applied in a volume of 100 ml/pot by irrigation, 20 days and 27 days after the transplantation of the soybean. Even in this case, the nutrient concentration was lower than that of the high soil nutrient groups (Groups 4 to 6).

TABLE 3

| | Inosine | Inoculation amount of | | |
|---|---|---|---|---|
| | concentration | root nodule bacterium | Fertilizer | Condition |
| Group 1 | 0 ppm | 4 × 10⁴ cfu/cm³ of soil | Only P₂O₅ and K₂O | Low soil |
| Group 2 | 2 ppm | 4 × 10⁴ cfu/cm³ of soil | Only P₂O₅ and K₂O | nutrient |
| Group 3 | 20 ppm | 4 × 10⁴ cfu/cm³ of soil | Only P₂O₅ and K₂O | |
| Group 4 | 0 ppm | 4 × 10⁴ cfu/cm³ of soil | Organic fertilizer | High soil |
| Group 5 | 2 ppm | 4 × 10⁴ cfu/cm³ of soil | Organic fertilizer | nutrient |
| Group 6 | 20 ppm | 4 × 10⁴ cfu/cm³ of soil | Organic fertilizer | |

TABLE 4

| | Element |
|---|---|
| | concentration (ppm) |
| CaCl₂ | Ca; 200 |

| | | |
|---|---|---|
| KPO | K; 97.5 | P; 77.5 |

| | |
|---|---|
| Fe-EDTA | Fe; 2.8 |
| MgSO₄•7H₂O | Mg; 30 |
| K₂SO₄ | K; 97.5 |
| CoCl₂•6H₂O | Co; 0.0295 |
| CuSO₄•5H₂O | Cu; 0.0635 |
| MnSO₄•4H₂O | Mn; 0.275 |
| ZnSO₄•7H₂O | Zn; 0.1635 |
| H₃BO₃ | B; 0.11 |
| Na₂MoO₂•2H₂O | Mo; 0.048 |

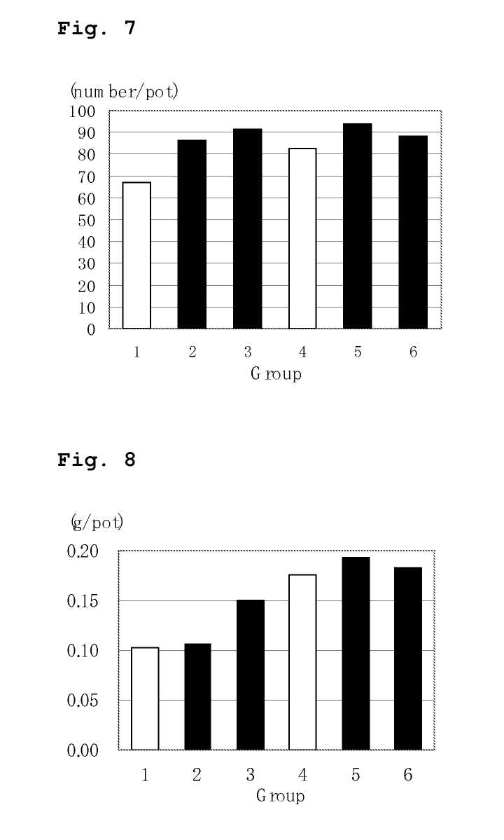

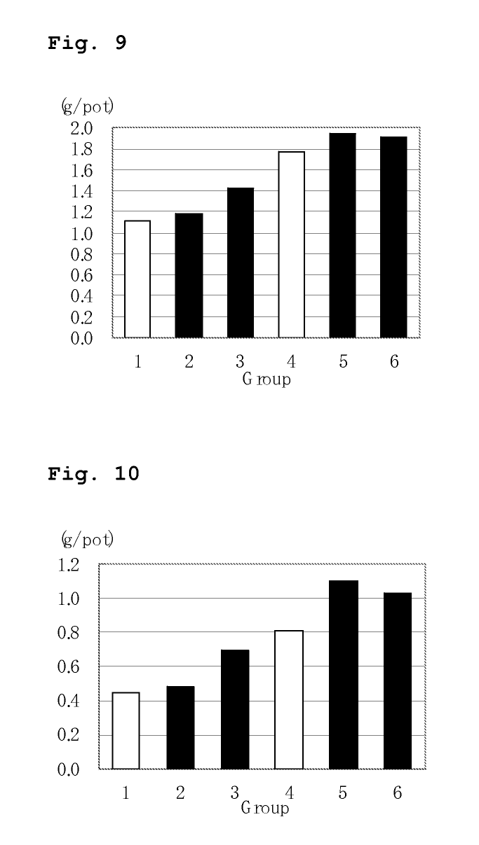

Thirty-three days after the seeding, the number of root nodules (FIG. 7), the dry weight of root nodules (FIG. 8), the dry weight of aboveground parts (FIG. 9), and the dry weight of underground parts (FIG. 10) were measured. Under the same soil fertilizer condition, the applications of the 2 ppm and 20 ppm inosine solutions both increased all of the number of root nodules, the dry weight of root nodules, the dry weight of aboveground parts and the dry weight of underground parts, as compared with the distilled water groups (0 ppm groups).

### Example 5

### Application of Nucleic Acids and Nucleic Acid Fermentation By-Product to Soybean by Irrigation

Soybean (Sachiyutaka) was seeded in 1.4 L of soil (vermiculite, moisture content: 50%) contained in each of pots (diameter: 15 cm). Calcium superphosphate and potassium sulfate were added as fertilizers, in amounts of 40 mg/pot in terms of P₂O₅ and K₂O, respectively. As a root nodule bacterium, “Mame-Zo” was applied to the soil at a root nodule bacterium concentration of 10 cfu/cm³ of the soil.

The treatment groups, as shown in Table 5, included a distilled water group (Group 1), hypoxanthine groups (Groups 2 and 3), guanosine groups (Groups 4 and 5), uracil groups (Group 6 and 7), and inosine fermentation by-product solution groups (Group 8 and 9). The inosine fermentation by-product solution contained 5.02 w/v % of inosine and 0.84 w/v % of hypoxanthine (1.65 w/v % in terms of inosine). One individual showing good growth before the development of a primary leaf was selected from each pot, and five individuals were used for each treatment group. Either distilled water or each of the solutions in a volume of 250 ml was applied by irrigation, 11, 19 and 24 days after the seeding. The hypoxanthine concentrations given as a one-time application were 0.18 ppm in the 1 ppm solution group and 1.8 ppm in the ppm solution group, which were equivalent to 141 g/ha and 1,412 g/ha, respectively. The guanosine concentrations and amounts given as a one-time application were 0.38 ppm and 297 g/ha in the 2.1 ppm solution group, and 3.75 ppm and 2,966 g/ha in the 21 ppm solution group, respectively. The uracil concentrations and amounts given as a one-time application were 0.15 ppm and 118 g/ha in the 0.84 ppm solution group, and 1.5 ppm and 1,186 kg/ha in the 8.4 ppm solution group, respectively. In the inosine fermentation by-product solution groups, inosine concentrations and amounts given as a one-time application were 0.5 ppm and 282 g/ha in the 30 ppm solution group, and 5 ppm and 2,825 g/ha in the 300 ppm solution group, in terms of inosine. In order to prevent deficiency of trace components, a solution having the composition shown in Table 4 was applied by irrigation in a volume of 200 ml/pot, 14, 21 and 28 days after the transplantation of the soybean.

TABLE 5

| | Test substance | Concentration | | Note |
|---|---|---|---|---|
| Group 1 | None | 0 | ppm | |
| Group 2 | Hypoxanthine | 1 | ppm | Equimolar to 2 ppm inosine |
| Group 3 | Hypoxanthine | 10 | ppm | Equimolar to 20 ppm inosine |
| Group 4 | Guanosine | 2.1 | ppm | Equimolar to 2 ppm inosine |
| Group 5 | Guanosine | 21 | ppm | Equimolar to 20 ppm inosine |
| Group 6 | Uracil | 0.84 | ppm | Equimolar to 2 ppm inosine |
| Group 7 | Uracil | 8.4 | ppm | Equimolar to 20 ppm inosine |
| Group 8 | Inosine fer- | 30 | ppm | Equimolar to 2 ppm inosine |
| | mentation | | | |
| | by-product | | | |
| | solution | | | |
| Group 9 | Inosine fer- | 300 | ppm | Equimolar to 20 ppm inosine |
| | mentation | | | |
| | by-product | | | |
| | solution | | | |

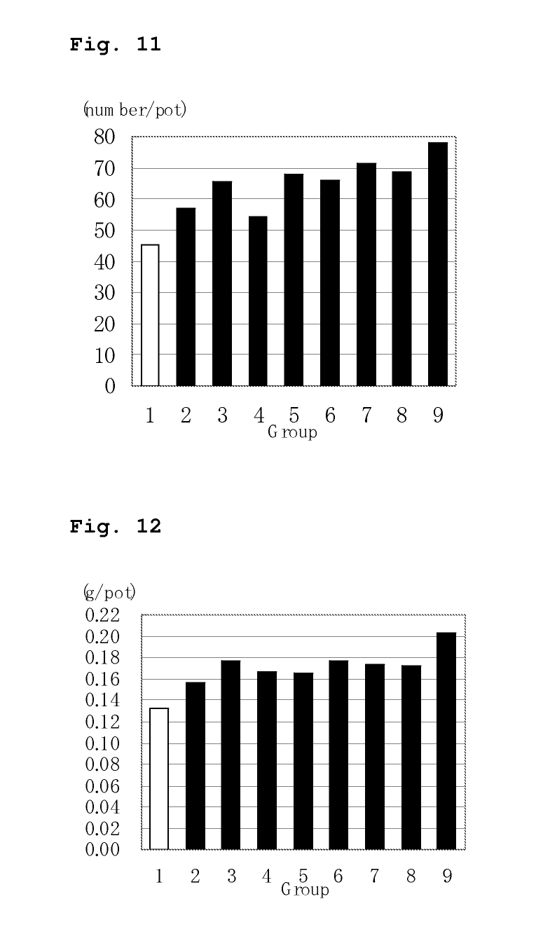

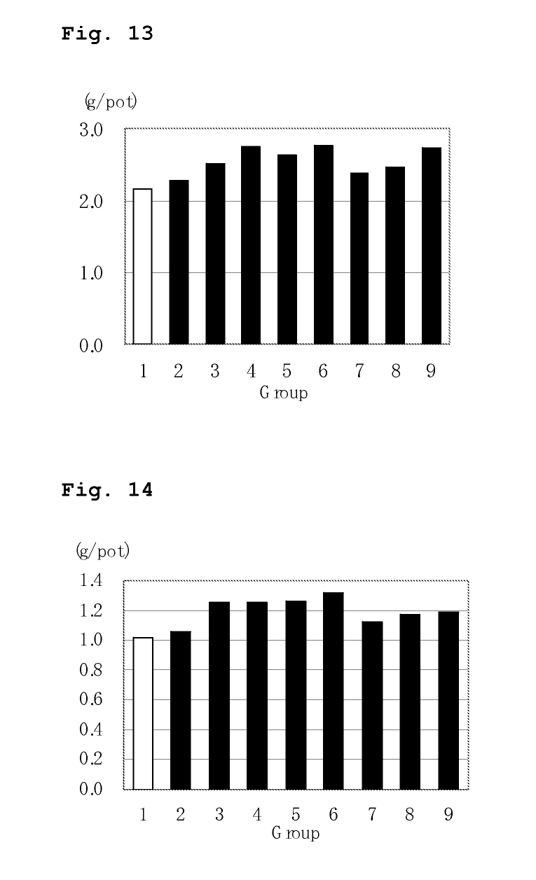

Forty days after the seeding, the number of root nodules (FIG. 11), the dry weight of root nodules (FIG. 12), the dry weight of aboveground parts (FIG. 13), and the dry weight of underground parts (FIG. 14) were measured. The application of each solution increased all of the number of root nodules, the dry weight of root nodules, the dry weight of aboveground parts and the dry weight of underground parts, as compared with the distilled water group.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

What is claimed is:

1. A composition for promoting root nodule formation of a leguminous plant, which comprises a compound selected from the group consisting of a nucleoside, a nucleotide, and a nucleobase, as an active ingredient.

2. The composition for promoting root nodule formation according to claim 1, which contains a fermentation by-product comprising the compound.

3. The composition for promoting root nodule formation according to claim 1, wherein the compound is selected from the group consisting of inosine, guanosine, uridine, inosinic acid, guanylic acid, uridylic acid, hypoxanthine, guanine, and uracil.

4. The composition for promoting root nodule formation according to claim 1, wherein the compound is selected from the group consisting of inosine, guanosine, hypoxanthine, and uracil.

5. The composition for promoting root nodule formation according to claim 1, which is applied to soil or a plant in an amount of 0.1 g to 20 kg/ha in terms of nucleoside.

6. The composition for promoting root nodule formation according to claim 1, which is in the form of a liquid containing 0.01 to 100 ppm of the compound in terms of nucleoside at the time of use.

7. A method for promoting root nodule formation of a leguminous plant, which comprises applying the composition for promoting root nodule formation according to claim 1, to soil or the plant.

8. The method according to claim 7, wherein the plant is a soybean plant.

9. The method according to claim 7, wherein the composition for promoting root nodule formation is applied to soil by spraying on a soil surface or by irrigation into soil, or to the plant by foliar spray.

10. The method according to claim 7, wherein the composition for promoting root nodule formation is applied in an amount of 0.1 g to 20 kg/ha in terms of nucleoside.

11. The method according to claim 7, wherein the composition for promoting root nodule formation is applied as an aqueous solution at a concentration of 0.01 to 100 ppm in terms of nucleoside.

12. The method according to claim 7, wherein the compound is selected from the group consisting of inosine, guanosine, uridine, inosinic acid, guanylic acid, uridylic acid, hypoxanthine, guanine, and uracil.

13. The method according to claim 7, wherein the compound is selected from the group consisting of inosine, guanosine, hypoxanthine, and uracil.
